(19) United States
(12) Patent Application Publication
(10) Pub. No.: US 2007/0129290 A1
(43) Pub. Date: Jun. 7, 2007

(54) Metabolite derivatives of the HDAC inhibitor FK228

(76) Inventors: Yat Sun Or, Watertown, MA (US); Gregory L. Verdine, Newton, MA (US); Mitchell Keegan, Marlborough, MA (US)

Correspondence Address: ELMORE PATENT LAW GROUP, PC, 209 MAIN STREET, N. CHELMSFORD, MA, 01863, US

(21) Appl. No.: 11/601,436

(22) Filed: Nov. 17, 2006

Related U.S. Application Data
(60) Provisional application No. 60/738,284, filed on Nov. 18, 2005.

(51) Int. Cl. A61K 38/12; C07K 5/12

(52) U.S. Cl. 514/10; 530/317

(57) ABSTRACT

The present invention relates to HDAC inhibitor derivatives, particularly derivatives of the free thiol of metabolites of the HDAC inhibitor FK228, pharmaceutical compositions thereof, and to methods of using such derivatives and pharmaceutical compositions thereof in the treatment of diseases associated with HDAC, in particular, tumor or cell proliferation diseases.

### RELATED APPLICATIONS

This application claims benefit of U.S. Provisional Application No. 60/738,284 filed on Nov. 18, 2005. The entire teachings of the above application is incorporated herein by reference.

### BACKGROUND OF THE INVENTION

Transcriptional regulation is a major event in cell differentiation, proliferation, and apoptosis. Transcriptional activation of a set of genes determines cell destination and for this reason transcription is tightly regulated by a variety of factors. One of its regulatory mechanisms involved in the process is an alteration in the tertiary structure of DNA, which affects transcription by modulating the accessibility of transcription factors to their target DNA segments. Nucleosomal integrity is regulated by the acetylation status of the core histones. In a hypoacetylated state, nucleosomes are tightly compacted and thus are nonpermissive for transcription. On the other hand, nucleosomes are relaxed by acetylation of the core histones, with the result being permissiveness to transcription. The acetylation status of the histones is governed by the balance of the activities of histone acetyl transferase (HAT) and histone deacetylase (HDAC).

HDAC inhibitors are likely to play an important role in the modulation of cellular proliferation. There are a wide variety of pathological cell proliferative conditions for which HDAC inhibitor therapeutics may be used in the treatment of diseases. For instance, HDAC inhibitors have been found to be useful in the treatment of cancer caused by the proliferation of neoplastic cells, such as tumors, neoplasms, carcinomas, sarcomas, leukemias, lymphomas and the like. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotrophic virus (HTLV) such as adult T-cell leukemia/ymphoma (ATLL), B-cell lymphoma, acute lymphocytic leukemia, acute nonlymphocytic leukemias, chronic lymphocytic leukemia, chronic myelogenous leukemia, acute myelogenous leukemia, Hodgkin's disease, non-Hodgkin's lymphomas, and multiple myeloma, myelodisplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal and esophageal), genito urinary cancers (e.g., prostate, bladder, renal, uterine, ovarian, testicular, rectal and colon), lung cancer, breast cancer, pancreatic cancer, melanoma and other skin cancers, stomach cancer, brain tumors, liver cancer and thyroid cancer.

HDAC inhibitors have also been found to be useful in the treatment and/or prevention of immune response or immune-mediated responses and diseases, such as the prevention or treatment of rejection following transplantation of synthetic or organic grafting materials, cells, organs or tissue to replace all or part of the function of tissues, such as heart, kidney, liver, bone marrow, skin, cornea, vessels, lung, pancreas, intestine, limb, muscle, nerve tissue, duodenum, small-bowel, pancreatic-islet-cell, including xeno-transplants, etc.; to treat or prevent graft-versus-host disease, autoimmune diseases, such as rheumatoid arthritis, systemic lupus erythematosus, thyroiditis, Hashimoto's thyroiditis, multiple sclerosis, myasthenia gravis, type I diabetes uveitis, juvenile-onset or recent-onset diabetes mellitus, uveitis, Graves disease, psoriasis, atopic dermatitis, Crohn's disease, ulcerative colitis, vasculitis, auto-antibody mediated diseases, aplastic anemia, Evan's syndrome, autoimmune hemolytic anemia, and the like; and further to treat infectious diseases causing abherent immune response and/or activation, such as traumatic or pathogen induced immune disregulation, including for example, that which are caused by hepatitis B and C infections, HIV, staphylococcus aureus infection, viral encephalitis, sepsis, parasitic diseases wherein damage is induced by an inflammatory response (e.g., leprosy); and to prevent or treat circulatory diseases, such as arteriosclerosis, atherosclerosis, vasculitis, polyarteritis nodosa and myocarditis. HDAC inhibitors may be used to prevent/suppress an immune response associated with a gene therapy treatment, such as the introduction of foreign genes into autologous cells and expression of the encoded product.

In addition, HDAC inhibitors have been found to be useful in the treatment of a variety of neurodegenerative diseases, a non-exhaustive list of which is: I. Disorders characterized by progressive dementia in the absence of other prominent neurologic signs, such as Alzheimer's disease; Senile dementia of the Alzheimer type; and Pick's disease (lobar atrophy); II. Syndromes combining progressive dementia with other prominent neurologic abnormalities such as A) syndromes appearing mainly in adults (e.g., Huntington's disease, Multiple system atrophy combining dementia with ataxia and/or manifestations of Parkinson's disease, Progressive supranuclear palsy (Steel-Richardson-Olszewski), diffuse Lewy body disease, and corticodentatonigral degeneration); and B) syndromes appearing mainly in children or young adults (e.g., Hallervorden-Spatz disease and progressive familial myoclonic epilepsy); III. Syndromes of gradually developing abnormalities of posture and movement such as paralysis agitans (Parkinson's disease), striatonigral degeneration, progressive supranuclear palsy, torsion dystonia (torsion spasm; dystonia musculorum deformans), spasmodic torticollis and other dyskinesis, familial tremor, and Gilles de la Tourette syndrome; IV. Syndromes of progressive ataxia such as cerebellar degenerations (e.g., cerebellar cortical degeneration and olivopontocerebellar atrophy (OPCA)); and spinocerebellar degeneration (Friedreich's atazia and related disorders); V. Syndrome of central autonomic nervous system failure (Shy-Drager syndrome); VI. Syndromes of muscular weakness and wasting without sensory changes (motomeuron disease such as amyotrophic lateral sclerosis, spinal muscular atrophy (e.g., infantile spinal muscular atrophy (Werdnig-Hoffman), juvenile spinal muscular atrophy (Wohlfart-Kugelberg-Welander) and other forms of familial spinal muscular atrophy), primary lateral sclerosis, and hereditary spastic paraplegia; VII. Syndromes combining muscular weakness and wasting with sensory changes (progressive neural muscular atrophy; chronic familial polyneuropathies) such as peroneal muscular atrophy (Charcot-Marie-Tooth), hypertrophic interstitial polyneuropathy (Dejerine-Sottas), and miscellaneous forms of chronic progressive neuropathy; VIII Syndromes of progressive visual loss such as pigmentary degeneration of the retina (retinitis pigmentosa), and hereditary optic atrophy (Leber's disease). Furthermore, HDAC inhibitors have been implicated in chromatin remodeling.

FK228 is a potent HDAC inhibitor and its identification and preparation is described in U.S. Pat. No. 4,977,138, incorporated herein by reference. FK228 is also known as FR901228, NSC630176 and depsipeptide and it has also been found to be a potent immunosuppressive. The following patents disclose some derivatives of FK228: WO 2005 0209134A1 (Yamanouchi Pharmaceutical Co., Ltd.), WO 2005 058298A2 (Fujisawa Pharmaceutical Co., Ltd.) and U.S. Pat. No. 6,548,479 (Skov, et. al.)

It has recently been reported that FK228 is a prodrug and undergoes disulfide bond reduction with GSH upon entering target cancer cells (Furumai et al. (2002) Cancer Res., 62:4916-4921). The reduction of the intramolecular disulfide bond of FK228 greatly enhanced its inhibitory activity and therefore, it is believed that the reduced compound is the active form.

It would be desirable to identify a derivative of FK228 that provides improved potency, improved desirable pharmacokinetic characteristics and improved adverse event profile of the FK228 and its metabolites previously identified for use as a therapeutic. The present invention meets this need and provides other related advantages.

### SUMMARY OF THE INVENTION

The present invention relates to HDAC inhibitor derivatives, particularly derivatives of the free thiol of metabolites of the HDAC inhibitor compound FK228, pharmaceutical compositions thereof, and to methods of using such derivatives and pharmaceutical compositions thereof in the treatment of disease.

### BRIEF DESCRIPTION OF THE FIGURES

FIG. 1 is the NMR spectra in vinyl region for FK228, metabolite A, product B/metabolite A mixture and product B acetate;

FIG. 2 is the analytical HPLC chromatograms and UV spectra of metabolites of A and product B acetate;

FIG. 3 is the HPLC chromatograms of product B;

FIG. 4 is the LC/MS chromatograms of product B;

FIG. 5 is an NMR spectrum of product B;

FIG. 6 is a 2D COSY spectrum of product B;

FIG. 7 is a UV spectrum of product B.

### DETAILED DESCRIPTION OF THE INVENTION

The instant invention provides compounds of formula (I) 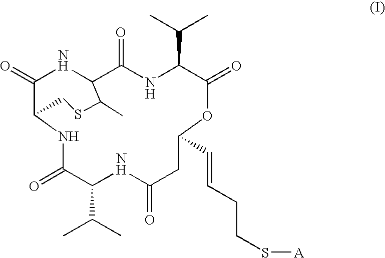
or their racemates, enantiomers, regioisomers, salts, esters or prodrugs thereof, wherein A is a moiety that can be cleaved in vivo to release a thiol group and includes, for example, aliphatic or aromatic acyl (to form an ester bond) and aliphatic or aromatic thioethers (to form a disulfide bond) and the like. Such aliphatic or aromatic groups can include a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, or a substituted or unsubstituted heterocyclic group.

In a preferred embodiment, A is —COR₁, —SC(═O)—O—R₁, or —SR₂. R₁ is independently hydrogen, a substituted or unsubstituted amino, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, or a substituted or unsubstituted heterocyclic group. In a preferred embodiment, R₁ is hydrogen, methyl, isobutyl, benzyl, bromobenzyl, substituted or unsubstituted aryl. R₂ is a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, or a substituted or unsubstituted heterocyclic group. In a preferred embodiment R₂ is methyl, ethyl, 2-hydroxyethyl, isobutyl, fatty acids, a substituted or unsubstituted benzyl, a substituted or unsubstituted aryl cysteine, homocysteine or glutathione.

In a preferred embodiment, A is also a moiety of formula I, thereby achieving a disulfide dimer of FK228.

The compounds of the invention are useful in the treatment of any disease in which inhibition of HDAC is desirable. The compounds of the invention may possess pharmaceutical and chemical properties which render them superior over FK228 and its metabolites as therapeutics. This invention, in addition to the compounds of formula 1, is intended to encompass the use of homologs and analogs of such compounds. In this context, homologs are molecules having substantial structural similarities to the above-described compounds and analogs are molecules having substantial biological similarities regardless of structural similarities.

The invention provides methods for treating cell proliferative diseases or conditions. The term “cell proliferative disease or condition” is meant to refer to any condition characterized by aberrant cell growth, preferably abnormally increased cellular proliferation. In one embodiment, the invention relates to a method of treating cancer in a subject in need of treatment comprising administering to said subject a therapeutically effective amount of a compound of Formula I. The term “cancer” refers to any cancer caused by the proliferation of neoplastic cells, such as tumors, neoplasms, carcinomas, sarcomas, leukemias, lymphomas and the like. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL), B-cell lymphoma, acute lymphocytic leukemia, acute nonlymphocytic leukemias, chronic lymphocytic leukemia, chronic 30 myelogenous leukemia, acute myelogenous leukemia, Hodgkin's disease, non-Hodgkin's lymphomas, and multiple myeloma, myelodisplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal and esophageal), genito urinary cancers (e.g., prostate, bladder, renal, uterine, ovarian, testicular, rectal and colon), lung cancer, breast cancer, pancreatic cancer, melanoma and other skin cancers, stomach cancer, brain tumors, liver cancer and thyroid cancer.

In another aspect, the invention provides the use of compounds of Formula I for the treatment and/or prevention of immune response or immune-mediated responses and diseases, such as the prevention or treatment of rejection following transplantation of synthetic or organic grafting materials, cells, organs or tissue to replace all or part of the function of tissues, such as heart, kidney, liver, bone marrow, skin, cornea, vessels, lung, pancreas, intestine, limb, muscle, nerve tissue, duodenum, small-bowel, pancreatic-islet-cell, including xeno-transplants, etc.; to treat or prevent graft-versus-host disease, autoimmune diseases, such as rheumatoid arthritis, systemic lupus erythematosus, thyroiditis, Hashimoto's thyroiditis, multiple sclerosis, myasthenia gravis, type I diabetes uveitis, juvenile-onset or recent-onset diabetes mellitus, uveitis, Graves disease, psoriasis, atopic dermatitis, Crohn's disease, ulcerative colitis, vasculitis, auto-antibody mediated diseases, aplastic anemia, Evan's syndrome, autoimmune hemolytic anemia, and the like; and further to treat infectious diseases causing abherent immune response and/or activation, such as traumatic or pathogen induced immune disregulation, including for example, that which are caused by hepatitis B and C infections, HIV, staphylococcus aureus infection, viral encephalitis, sepsis, parasitic diseases wherein damage is induced by an inflammatory response (e.g., leprosy); and to prevent or treat circulatory diseases, such as arteriosclerosis, atherosclerosis, vasculitis, polyarteritis nodosa and myocarditis. In addition the present invention may be used to prevent/suppress an immune response associated with a gene therapy treatment, such as the introduction of foreign genes into autologous cells and expression of the encoded product. Thus in one embodiment, the invention relates to a method of treating an immune response disease or disorder or an immune-mediated response or disorder in a subject in need of treatment comprising administering to said subject a therapeutically effective amount of a compound of Formula I.

In another aspect, the invention provides the use of compounds of Formula I in the treatment of a variety of neurodegenerative diseases, a non-exhaustive list of which is: I. Disorders characterized by progressive dementia in the absence of other prominent neurologic signs, such as Alzheimer's disease; Senile dementia of the Alzheimer type; and Pick's disease (lobar atrophy); II. Syndromes combining progressive dementia with other prominent neurologic abnormalities such as A) syndromes appearing mainly in adults (e.g., Huntington's disease, Multiple system atrophy combining dementia with ataxia and/or manifestations of Parkinson's disease, Progressive supranuclear palsy (Steel-Richardson-Olszewski), diffuse Lewy body disease, and corticodentatonigral degeneration); and B) syndromes appearing mainly in children or young adults (e.g., Hallervorden-Spatz disease and progressive familial myoclonic epilepsy); III. Syndromes of gradually developing abnormalities of posture and movement such as paralysis agitans (Parkinson's disease), striatonigral degeneration, progressive supranuclear palsy, torsion dystonia (torsion spasm; dystonia musculorum deformans), spasmodic torticollis and other dyskinesis, familial tremor, and Gilles de la Tourette syndrome; IV. Syndromes of progressive ataxia such as cerebellar degenerations (e.g., cerebellar cortical degeneration and olivopontocerebellar atrophy (OPCA)); and spinocerebellar degeneration (Friedreich's atazia and related disorders); V. Syndrome of central autonomic nervous system failure (Shy-Drager syndrome); VI. Syndromes of muscular weakness and wasting without sensory changes (motomeuron disease such as amyotrophic lateral sclerosis, spinal muscular atrophy (e.g., infantile spinal muscular atrophy (Werdnig-Hoffman), juvenile spinal muscular atrophy (Wohlfart-Kugelberg-Welander) and other forms of familial spinal muscular atrophy), primary lateral sclerosis, and hereditary spastic paraplegia; VII. Syndromes combining muscular weakness and wasting with sensory changes (progressive neural muscular atrophy; chronic familial polyneuropathies) such as peroneal muscular atrophy (Charcot-Marie-Tooth), hypertrophic interstitial polyneuropathy (Dejerine-Sottas), and miscellaneous forms of chronic progressive neuropathy; VIII Syndromes of progressive visual loss such as pigmentary degeneration of the retina (retinitis pigmentosa), and hereditary optic atrophy (Leber's disease). Furthermore, HDAC inhibitors have been implicated in chromatin remodeling.

The invention encompasses pharmaceutical compositions comprising pharmaceutically acceptable salts of the compounds of the invention as described above. The invention also encompasses pharmaceutical compositions comprising hydrates of the compounds of the invention. The term “hydrate” includes but is not limited to hemihydrate, monohydrate, dihydrate, trihydrate and the like. The invention further encompasses pharmaceutical compositions comprising any solid or liquid physical form of the compound of the invention. For example, the compounds can be in a crystalline form, in amorphous form, and have any particle size. The particles may be micronized, or may be agglomerated, particulate granules, powders, oils, oily suspensions or any other form of solid or liquid physical form.

The compounds of the invention, and derivatives, fragments, analogs, homologs pharmaceutically acceptable salts or hydrate thereof can be incorporated into pharmaceutical compositions suitable for administration, together with a pharmaceutically acceptable carrier or excipient. Such compositions typically comprise a therapeutically effective amount of any of the compounds above, and a pharmaceutically acceptable carrier. Preferably, the effective amount when treating cancer is an amount effective to selectively induce terminal differentiation of suitable neoplastic cells and less than an amount which causes toxicity in a patient.

HDAC inhibitors or HDAC prodrugs may be administered by any suitable means, including, without limitation, parenteral, intravenous, intramuscular, subcutaneous, implantation, oral, sublingual, buccal, nasal, pulmonary, transdermal, topical, vaginal, rectal, and transmucosal administrations or the like. Pharmaceutical preparations include a solid, semisolid or liquid preparation (tablet, pellet, troche, capsule, suppository, cream, ointment, aerosol, powder, liquid, emulsion, suspension, syrup, injection etc.) containing a histone deacetylase inhibitor as an active ingredient, which is suitable for selected mode of administration. In one embodiment, the pharmaceutical compositions are administered orally, and are thus formulated in a form suitable for oral administration, i.e., as a solid or a liquid preparation. Suitable solid oral formulations include tablets, capsules, pills, granules, pellets, sachets and effervescent, powders, and the like. Suitable liquid oral formulations include solutions, suspensions, dispersions, emulsions, oils and the like. In one embodiment of the present invention, the composition is formulated in a capsule. In accordance with this embodiment, the compositions of the present invention comprise in addition to the active compound and the inert carrier or diluent, a hard gelatin capsule.

Any inert excipient that is commonly used as a carrier or diluent may be used in the formulations of the present invention, such as for example, a gum, a starch, a sugar, a cellulosic material, an acrylate, or mixtures thereof. A preferred diluent is microcrystalline cellulose. The compositions may further comprise a disintegrating agent (e.g., croscarmellose sodium) and a lubricant (e.g., magnesium stearate), and in addition may comprise one or more additives selected from a binder, a buffer, a protease inhibitor, a surfactant, a solubilizing agent, a plasticizer, an emulsifier, a stabilizing agent, a viscosity increasing agent, a sweetener, a film forming agent, or any combination thereof. Furthermore, the compositions of the present invention may be in the form of controlled release or immediate release formulations.

For liquid formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil. Solutions or suspensions can also include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or extrose. The pH can be adjusted with acids or bases, such as hydrochloric acid or sodium hydroxide.

In addition, the compositions may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate, Primogel), buffers (e.g., tris-HCI., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol), a glidant (e.g., colloidal silicon dioxide), anti-oxidants (e.g., ascorbic acid, sodium metabisulfite, butylated hydroxyanisole), stabilizers (e.g., hydroxypropyl cellulose, hyroxypropylmethyl cellulose), viscosity increasing agents (e.g., carbomer, colloidal silicon dioxide, ethyl cellulose, guar gum), sweeteners (e.g., sucrose, aspartame, citric acid), flavoring agents (e.g., peppermint, methyl salicylate, or orange flavoring), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates) and/or adjuvants.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat No. 4,522,811.

It is especially advantageous to formulate oral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

The daily administration is then repeated continuously for a period of several days to several years. Oral treatment may continue for between one week and the life of the patient. Preferably the administration takes place for five consecutive days after which time the patient can be evaluated to determine if further administration is required. The administration can be continuous or intermittent, i.e., treatment for a number of consecutive days followed by a rest period. The compounds of the present invention may be administered intravenously on the first day of treatment, with oral administration on the second day and all consecutive days thereafter.

The preparation of pharmaceutical compositions that contain an active component is well understood in the art, for example, by mixing, granulating, or tablet-forming processes. The active therapeutic ingredient is often mixed with excipients that are pharmaceutically acceptable and compatible with the active ingredient. For oral administration, the active agents are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions and the like as detailed above.

The amount of the compound administered to the patient is less than an amount that would cause toxicity in the patient. In the certain embodiments, the amount of the compound that is administered to the patient is less than the amount that causes a concentration of the compound in the patient's plasma to equal or exceed the toxic level of the compound. Preferably, the concentration of the compound in the patient's plasma is maintained at about 10 nM. In another embodiment, the concentration of the compound in the patient's plasma is maintained at about 25 nM. In another embodiment, the concentration of the compound in the patient's plasma is maintained at about 50 nM. In another embodiment, the concentration of the compound in the patient's plasma is maintained at about 100 nM. In another embodiment, the concentration of the compound in the patient's plasma is maintained at about 500 nM. In another embodiment, the concentration of the compound in the patient's plasma is maintained at about 1000 nM. In another embodiment, the concentration of the compound in the patient's plasma is maintained at about 2500 nM. In another embodiment, the concentration of the compound in the patient's plasma is maintained at about 5000 nM. The optimal amount of the compound that should be administered to the patient in the practice of the present invention will depend on the particular compound used and the type of cancer being treated.

HDAC inhibitors or HDAC prodrugs may be used in combination with other drug therapies, including, but not limited to, demethylating agents (decitabine, 5-azacitidine), clofarabine, fludarabine, cladribine, rituximab (Rituxan), Mylotarg and Gleevec. The HDAC inhibitors can be administered simultaneously (as a single preparation or separate preparation) or sequentially to the other drug therapy. In general, a combination therapy envisions administration of two or more drugs during a single cycle or course of therapy.

Definitions

Listed below are definitions of various terms used to describe this invention. These definitions apply to the terms as they are used throughout this specification and claims, unless otherwise limited in specific instances, either individually or as part of a larger group.

An “aliphatic group” is non-aromatic moiety that may contain any combination of carbon atoms, hydrogen atoms, halogen atoms, oxygen, nitrogen or other atoms, and optionally contain one or more units of unsaturation, e.g., double and/or triple bonds. An aliphatic group may be straight chained, branched or cyclic and preferably contains between about 1 and about 24 carbon atoms, more typically between about 1 and about 12 carbon atoms. In addition to aliphatic hydrocarbon groups, aliphatic groups include, for example, polyalkoxyalkyls, such as polyalkylene glycols, polyamines, and polyimines, for example. Such aliphatic groups may be further substituted by one or more aliphatic substituents.

The terms “aryl” or “aromatic,” as used herein, refer to a mono- or bicyclic carbocyclic ring system having one or two aromatic rings including, but not limited to, phenyl, naphthyl, tetrahydronaphthyl, indanyl, idenyl and the like.

The terms “substituted aryl” or “substituted aromatic” as used herein, refer to an aryl group, as previously defined, substituted by one, two, three or more aromatic substituents.

The terms “heteroaryl or “heteroaromatic,” as used herein, refer to a mono-, bi-, or tri-cyclic aromatic radical or ring having from five to ten ring atoms of which at least one ring atom is selected from S, O and N; zero, one, two, three or more ring atoms are additional heteroatoms independently selected from S, O and N; and the remaining ring atoms are carbon, wherein any N or S contained within the ring may be optionally oxidized. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, tetrazolyl and the like. The heteroaromatic ring may be bonded to the chemical structure through a carbon or hetero atom.

The terms “substituted heteroaryl” or “substituted heteroaromatic,” as used herein, refer to a heteroaryl group as previously defined, substituted by one, two, three or more aromatic substituents.

The term “alicyclic,” as used herein, denotes a monovalent group derived from a monocyclic or bicyclic saturated carbocyclic ring compound by the removal of a single hydrogen atom. Examples include, but not limited to, cyclopropyl, cyclobutyl, cyclopentyl, cyclohexyl, bicyclo [2.2.1]heptyl, and bicyclo [2.2.2]octyl.

The term “substituted alicyclic” group as previously defined, substituted by one, two, three or more aliphatic substituents.

The terms “heterocyclic” as used herein, refers to a non-aromatic 5-, 6- or 7-membered ring or a bi- or tri-cyclic group fused system, where (i) each ring contains between one and three heteroatoms independently selected from oxygen, sulfur and nitrogen, (ii) each 5-membered ring has 0 to 1 double bonds and each 6-membered ring has 0 to 2 double bonds, (iii) the nitrogen and sulfur heteroatoms may optionally be oxidized, (iv) the nitrogen heteroatom may optionally be quaternized, (iv) any of the above rings may be fused to a benzene ring, and (v) the remaining ring atoms are carbon atoms which may be optionally oxo-substituted. Representative heterocycloalkyl groups include, but are not limited to, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, tetrahydrofuryl, and the like.

The term “substituted heterocyclic,” as used herein, refers to a heterocyclic group, as previously defined, substituted by one, two, three or more aliphatic substituents.

Suitable aliphatic or aromatic substituents include, but are not limited to, —F, —Cl, —Br, —I , —OH, protected hydroxy, aliphatic ethers, aromatic ethers, oxo, —NO₂, —CN, —C₁-C₁₂-alkyl optionally substituted with halogen (such as perhaloalkyls), C₂-C₁₂-alkenyl optionally substituted with halogen, —C₂-C₁₂-alkynyl optionally substituted with halogen, —NH₂, protected amino, —NH—C₁-C₁₂-alkyl, —NH—C₂-C₁₂-alkenyl, —NH—C₂-C₁₂-alkynyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₁₂-alkenyl, —O—C₂-C₁₂-alkynyl, —O—C₃-C₁₂-cycloalkyl, —O-aryl, —O-heteroaryl, —O-heterocycloalkyl, —C(O)—C₁-C₁₂-alkyl, —C(O)—C₂-C₁₂-alkenyl, —C(O)—C₂-C₁₂-alkynyl, —C(O)—C₃-C₁₂-cycloalkyl, —C(O)-aryl, —C(O)-heteroaryl, —C(O)-heterocycloalkyl, —CONH₂, —CONH—C₁-C₁₂-alkyl, —CONH—C₂-C₁₂-alkenyl, —CONH—C₂-C₁₂-alkynyl, —CONH-C₃-C₁₂-cycloalkyl, —CONH-aryl, —CONH-heteroaryl, —CONH-heterocycloalkyl, —CO₂—C₁-C₁₂-alkyl, —CO₂—C₂-C₁₂-alkenyl, —CO₂—C₂-C₁₂-alkynyl, —CO₂—C₃-C₁₂-cycloalkyl, —CO₂-aryl, —CO₂-heteroaryl, —CO₂-heterocycloalkyl, —OCO₂—C₁-C₁₂-alkyl, —OCO₂—C₂-C₁₂-alkenyl, —OCO₂—C₂-C₁₂-alkynyl, —OCO₂—C₃-C₁₂-cycloalkyl, -—OCO₂-aryl, —OCO₂-heteroaryl, —OCO₂-heterocycloalkyl, —OCONH₂, —OCONH—C₁-C₁₂-alkyl, —OCONH—C₂-C₁₂-alkenyl, —OCONH—C₂-C₁₂-alkynyl, —OCONH—C₃-C₁₂-cycloalkyl, —OCONH-aryl, —OCONH-heteroaryl, —OCONH-heterocycloalkyl, —NHC(O)—C₁-C₁₂-alkyl, —NHC(O)—C₂-C₁₂-alkenyl, —NHC(O)—C₂-C₁₂-alkynyl, —NHC(O)—C₃-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₁₂-alkenyl, —NHCO₂—C₂-C₁₂-alkynyl, —NHCO₂—C₃-C₁₂-cycloalkyl, —NHCO₂-aryl, —NHCO₂-heteroaryl, —NHCO₂-heterocycloalkyl, —NHC(O)NH₂, NHC(O)NH—C₁-C₁₂-alkyl, —NHC(O)NH—C₂-C₁₂-alkenyl, —NHC(O)NH—C₂-C₁₂-alkynyl, —NHC(O)NH—C₃-C₁₂-cycloalkyl, —NHC(O)NH-aryl, —NHC(O)NH-heteroaryl, —NHC(O)NH-heterocycloalkyl, NHC(S)NH₂, NHC(S)NH—C₁-C₁₂-alkyl, —NHC(S)NH—C₂-C₁₂-alkenyl, —NHC(S)NH—C₂-C₁₂-alkynyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, NHC(NH)NH—C₁-C₁₂-alkyl, —NHC(NH)NH—C₂-C₁₂-alkenyl, —NHC(NH)NH—C₂-C₁₂-alkynyl, —NHC(NH)NH—C₃-C₁₂-cycloalkyl, —NHC(NH)NH-aryl, —NHC(NH)NH-heteroaryl, —NHC(NH)NH-heterocycloalkyl, NHC(NH)—C₁-C₁₂-alkyl, —NHC(NH)—C₂-C₁₂-alkenyl, —NHC(NH)—C₂-C₁₂-alkynyl, —NHC(NH)—C₃-C₁₂-cycloalkyl, —NHC(NH)-aryl, —NHC(NH)-heteroaryl, —NHC(NH)-heterocycloalkyl, —C(NH)NH—C₁-C₁₂-alkyl, —C(NH)NH—C₂-C₁₂-alkenyl, —C(NH)NH—C₂-C₁₂-alkynyl, —C(NH)NH—C₃-C₁₂-cycloalkyl, —C(NH)NH-aryl, —C(NH)NH-heteroaryl, —C(NH)NH-heterocycloalkyl, —S(O)—C₁-C₁₂-alkyl, —S(O)—C₂-C₁₂-alkenyl, —S(O)—C₂-C₁₂-alkynyl, —S(O)—C₃-C₁₂-cycloalkyl, —S(O)-aryl, —S(O)-heteroaryl, —S(O)-heterocycloalkyl —SO₂NH₂, —SO₂NH—C₁-C₁₂-alkyl, —SO₂NH—C₂-C₁₂-alkenyl, —SO₂NH—C₂-C₁₂-alkynyl, —SO₂NH—C₃-C₁₂-cycloalkyl, —SO₂NH-aryl, —SO₂NH-heteroaryl, —SO₂NH-heterocycloalkyl, —NHSO₂—C₁-C₁₂-alkyl, —NHSO₂—C₂-C₁₂-alkenyl, —NHSO₂—C₂-C₁₂-alkynyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₁₂-alkenyl, —S—C₂-C₁₂-alkynyl, —S—C₃-C₁₂-cycloalkyl, —S-aryl, —S-heteroaryl, —S-heterocycloalkyl, or methylthiomethyl. It is understood that the aryls, heteroaryls, alkyls and the like can be further substituted.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid or inorganic acid. Examples of pharmaceutically acceptable nontoxic acid addition salts include, but are not limited to, salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid lactobionic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present invention. “Prodrug”, as used herein means a compound which is convertible in vivo by metabolic means (e.g. by hydrolysis) to a compound of Formula I. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991); Bundgaard, et al., Journal of Drug Deliver Reviews, 8:1-38(1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

As used herein, “pharmaceutically acceptable carrier” is intended to include any and all solvents, dispersion media, coatings, antibacterial and antifungal agents, isotonic and absorption delaying agents, and the like, compatible with pharmaceutical administration, such as sterile pyrogen-free water. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Synthetic Methods

The compounds and processes of the present invention will be better understood in connection with the synthetic schemes 1, that illustrates the methods by which the compounds of the invention may be prepared. As shown in general scheme 1, FK228 is first reduced to compound 1-1, a metabolite of FK228. An internal thiol Michael addition results in compound 1-2 can be obtained without isolating intermediate (1-1). The free thiol may be derivatized further to yield a compound of Formula 1 (1-3).

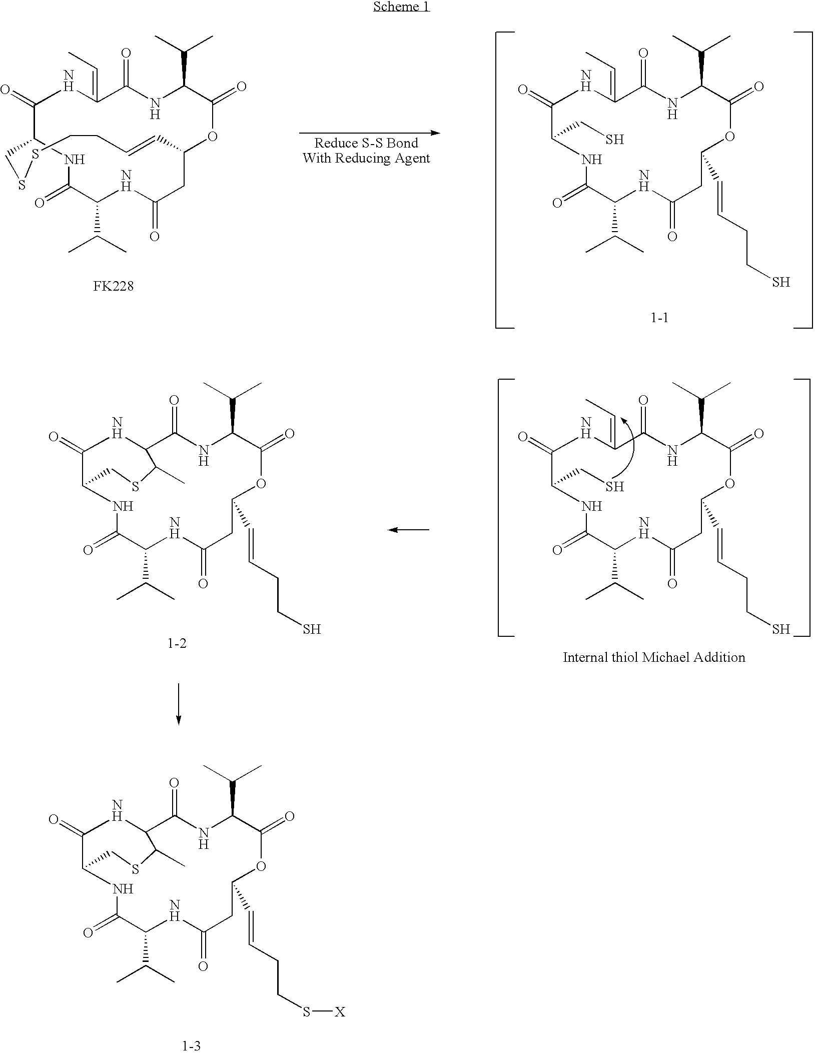

As shown in scheme 2, the reduction of FK228 via a hydride reducing agent, such as but not limited to NaBH₄, PS-BH₄, n-Bu₄NBH₄, n-butyl tin hydride, LiAlH(O-tert-Bu)₃, PS-PPh₂, Ph₃P, nBU₃P, or Et₃P yields compound 1-2. Treatment of the resulting compound 1-2 with an appropriate carboxylic acid using standard ester coupling reagent such as DCC, HATU, BOPCl and the like yields 10 the desired thioesters 2-1.

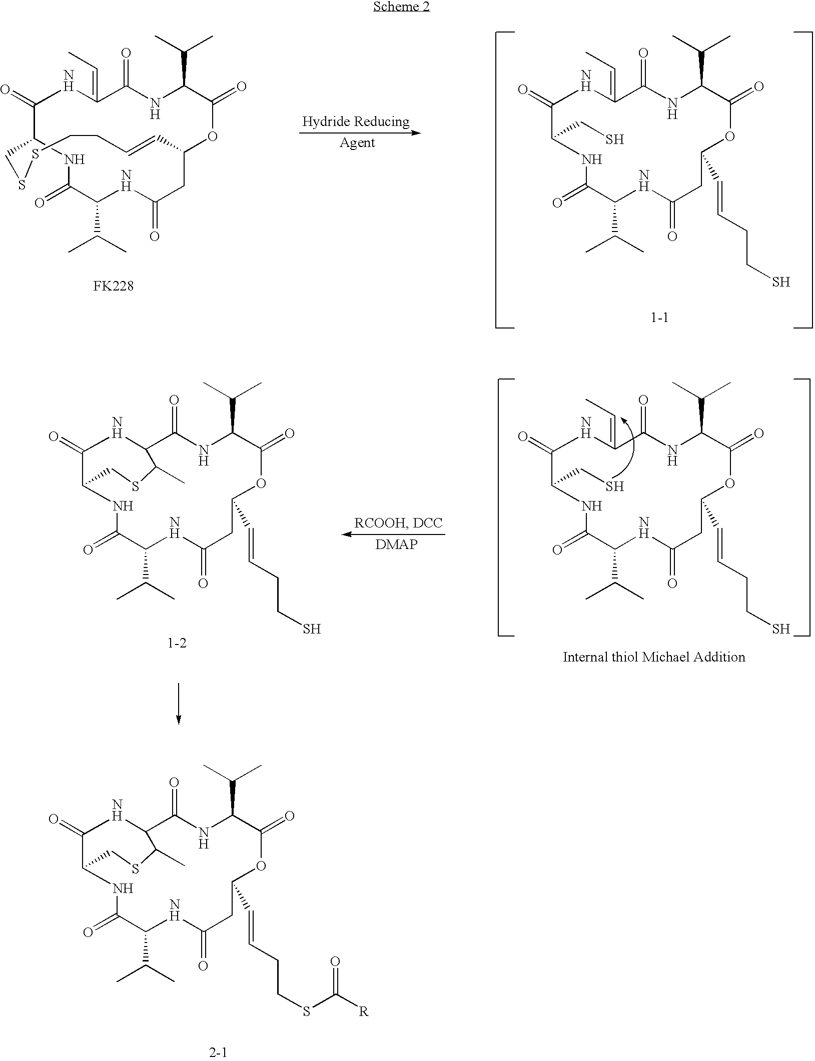

Scheme 3 shows the formation of thioether compounds (3-1) from intermediate (1-2) Scheme with alkyl or substituted alkyl halide.

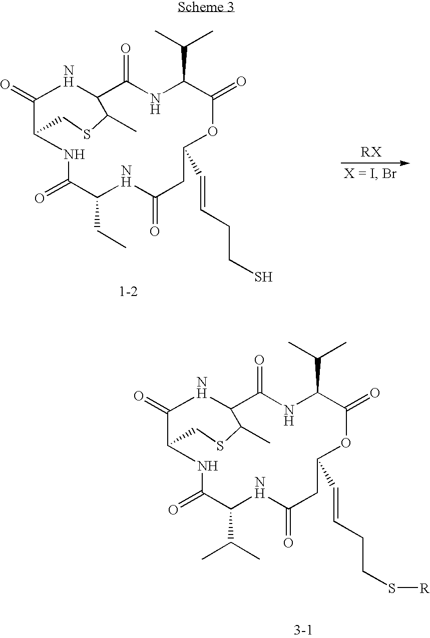

Scheme 4 shows the formation of the disulfide compounds (4-2) directly from FK228 with an appropriate substituted and unsubstituted thiol under a buffer condition (see Houk, J. et. al., J. Am. Chem Soc. 1987, 109, 6825-6836). When R contains an acid moiety, the thiol fatty acid can be obtained from the corresponding halogenated alkyl acid with 4-methoxy trityl thiol (see Barlos, K. et. al. Tet. Lett., 2001, 42, 6965-6967 for further details).

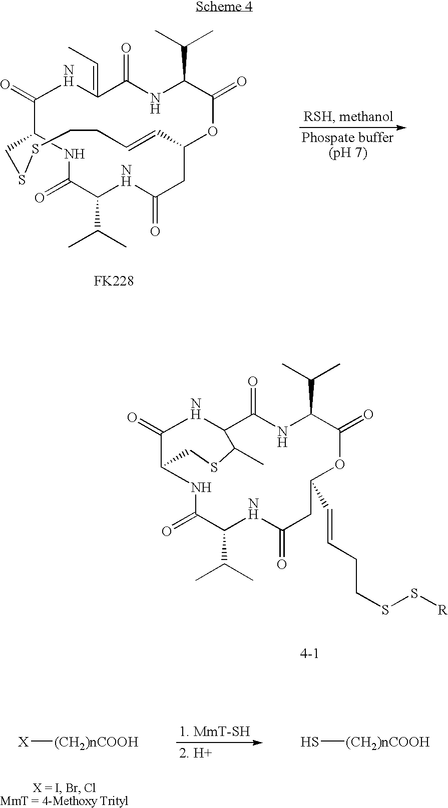

Scheme 5 describes the formation of FK228 disulfide dimer (5-1) from common intermediate (1-2) with an oxidizing agent such as, but not limited to, air or iodine.

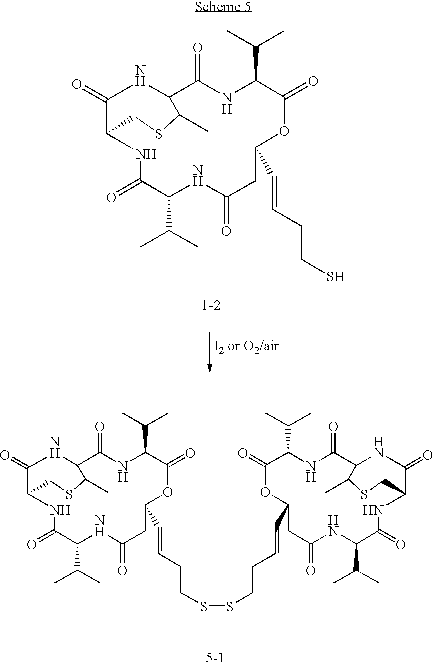

### EXAMPLES

The compounds and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those 10 relating to the chemical structures, substituents, derivatives, formulations and/or methods of the invention may be made without departing from the spirit of the invention and the scope of the appended claims.

### Example

### 1. Thioesters

Formation of Product C Thioesters via Hydride Reduction

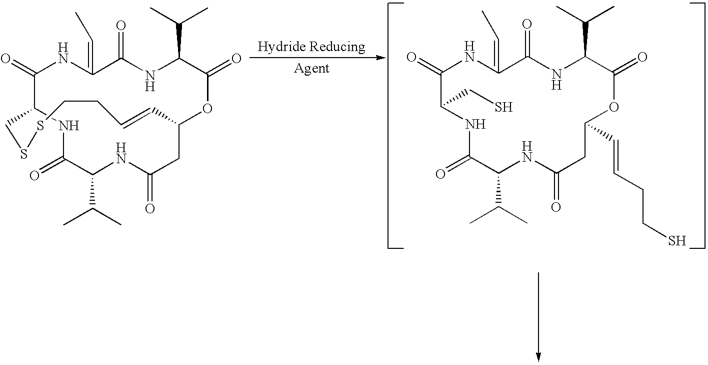

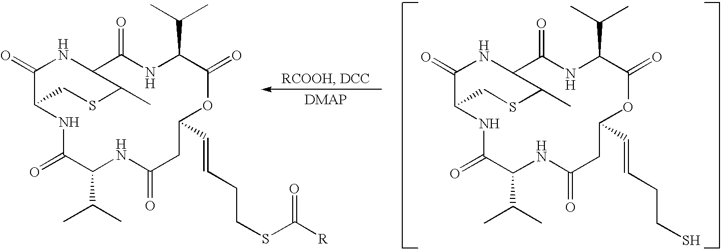

| Hydride Reducing Agent | Carboxylic Acid |
|---|---|
| PS—BH₄ | Formic |
| NaBH₄ | Acetic |
| n-Bu₄NBH₄ | Isobutyric |
| n-Bu₃SnH | Benzoic |
| LiAlH(O-tert-Bu)₃ | p-Bromobenzoic |
| | Phenylacetic, |
| | and 4-8 other |
| | carboxylic acids |

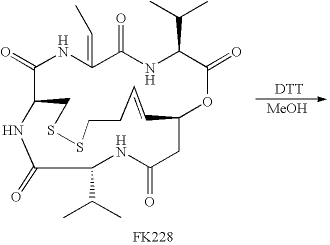

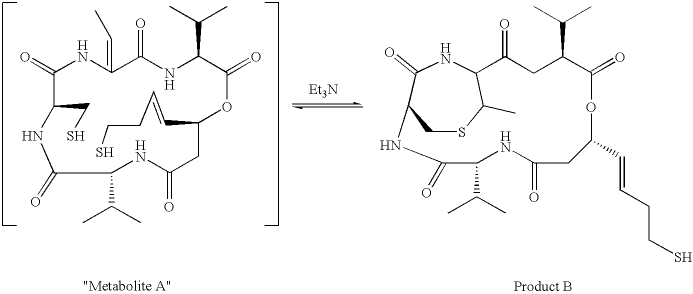

Reduction of FK228 with DTT (3.5 eq) in methanol (MeOH) is relatively clean providing mostly Metabolite A (90.6%, R_{T}9.0 min) and small amounts of Metabolite A dimers (R_{T}S 13.3 min and 14.0 min), but no Product B. Prolonged reaction time (weekend) results in the formation of small amounts of other reduction products [R_{T}S 8.4 min (9%) and 8.7 min (16%)], but no Product B.

When Metabolite A is isolated and then treated with triethylamine (Et₃N) in MeOH, the principal product (35.6%) is Product B (R_{T} 9.0 min) with some (31.1%) unreacted Metabolite A (R_{T}9.4 min), along with some FK228 (5.5%; absence of DTT), another component at 9.2 min (14.1%), and small amounts (2.9%-5.6%) of dimeric products. The components at 9.2 min and 10.7 min are unique in that they do not have any significant UV absorption at 225 nm (from loss of conjugated double bond), suggesting that both are products resulting from internal Michael addition like that which gives Product B; the component at 9.2 min is, however, always a minor component (usually less than 4%). Whenever Metabolite A is treated with Et₃N, the component at 10.7 min (i.e., Product B) is always the major product.

The cleanest and fastest way to produce Product B involves simultaneously treating FK228 with both DTT and Et₃N in MeOH. This results in rapid and complete reduction to Metabolite A, followed by cyclization to Product B. This cyclization may be reversible and results in what is likely an equilibrium mixture of Metabolite A (37.7%) and Product B (48.3%) along with smaller amounts of components at 9.2 min (3.1%; this is the other possible Michael adduct discussed above) and 14.3 min (6.5%).

That the product at 10.7 min is Product B is substantiated by ¹H NMR of the Product B/Metabolite A mixture which shows 70% reduction in the integration for the quartet at δ 6.8 (the signal for the vinyl proton of the conjugated double bond) and an upfield shift of the doublet at δ 1.82 (vinylic methyl group) to δ 1.51 (non-vinylic methyl group) as predicted (using NMR software), observance of the lack of an absorption maximum at 225 nm (in contrast to Metabolite A and FK228), and the observation that acetylation results only in mono-acetylation.

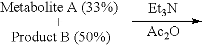

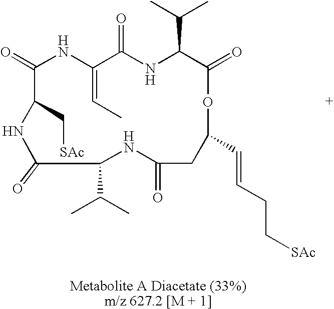

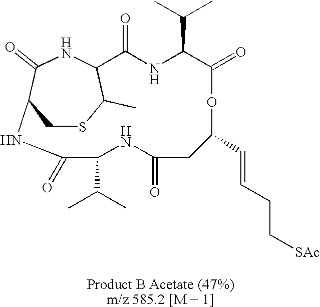

HPLC/MS Data
HPLC method
Column: Phenomenex Synergi 4μ Fusion-RP 80(150 mm × 4.6 mm)
Run time: 20 min
Post run time: 6 min
Flow rate: 1.5 mL/min
Injector volume: 5 μL
Time table for gradient:
0 min 20% ACN
15 min 60% ACN
19 min 100% ACN
20 min 100% ACN
HPLC Peak assignments

| Component | R_{T} | Identification | Mass |
|---|---|---|---|
| 1* | 8.4 min | — | 543.3 |
| 2* | 8.7 min | — | 543.2 |
| 3 | 9.0 min | Metabolite A | 543.2 |
| 4** | 9.2 min | — | 543.3 |
| 5 | 9.4 min | FK228 | 541.3 |
| 5 | 9.7 min | Metabolite A monoacetate | 585.1 |
| 6*** | 10.1 min | — | 543.3 |
| 7 | 10.3 min | Metabolite A diacetate | 627.2 |
| 8*** | 10.3 min | — | 543.3 |
| 9 | 10.7 min | Product B | 543.2 |
| 10 | 11.0 min | Product B acetate | 585.1 |
| 11 | 13.3 min | Metabolite A dimer #2 | 1083.3 |
| 12 | 14.0 min | Metabolite A dimer #1 | 1083.5 |

*Formed only, and in small amounts, from prolonged time for reduction of FK228.
**A Michael adduct like Product B (see next page for possible structures).
***Formed only when mixtures of Metabolite A containing small amounts of components 1 and 2 was treated with triethylamine.

While Product B Acetate does not undergo S—C(O) bond cleavage in methanol alone (whether at room temperature or at 50° C.), the additional presence of base (ammonia or triethylamine) results in the rapid conversion to an equilibrium mixture of primarily two components, Product B and Metabolite A, in proportions similar to those obtained when FK228 is treated with DTT in the presence of methanol and triethylamine (i.e., 33-39% Metabolite A and 38-50% Product B). Other conditions (NH₃ and polymer-supported borohydride) involving non-protic solvents (DCM, EtOAc, or THF) did not successfully cleave the S —C(O) bond.

A freshly prepared solution (from FK228/DTT/MeOH/Et₃N) containing Product B (46-50%, R_{T}10.7 min) and Metabolite A (33-38%, R_{T}9.0 min) always contains two impurities (reduced FK228-DTT adducts) which elute between Product B and Metabolite A [R_{T}9.7 min (2%), R_{T}9.9 min (3%)]. These two impurities grow over time (whilte Product B decreases), especially when the mixture is evaporated to an oil. In an attempt to prepare a Product B/Metabolite A mixture that would be free of these two impurities, reactions were run with FK228 in methanol using polymer-bound DTT, with and without triethylamine; however, the polymer-bound DTT behaved very different from free DTT with no apparent reduction, producing only FK228 dimers and trimers lacking free thiol groups. Hence, Product B-Metabolite A mixtures must be freshly prepared, just prior to chromatographic purification.

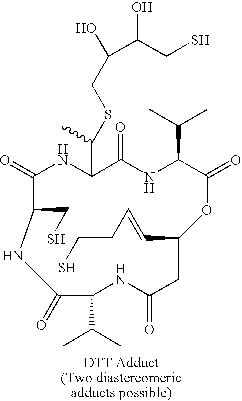

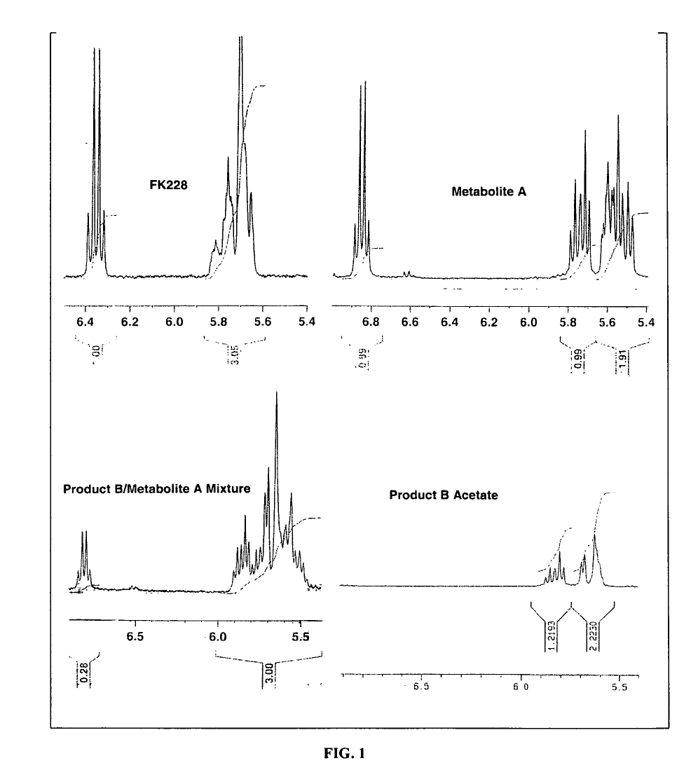

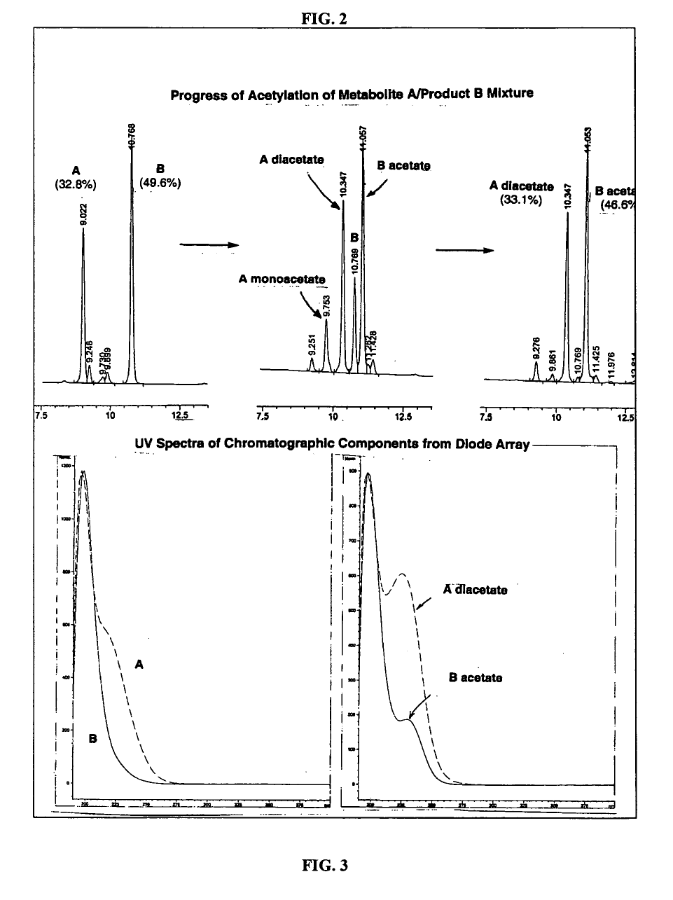

The vinyl region of the NMR spectra of FK228, Metabolite A, Product B, and Product B Acetate is shown FIG. 1. As you can see, the quartet at δ 6.3-6.9 (present in both FK228 and Metabolite A) is greatly diminished in the Product B-Metabolite A mixture and is totally absent in purified Product B Acetate. That this quartet is indeed the enamide vinyl proton is evidenced by the fact that it is coupled to the doublet (δ 1.82) of the adjacent methyl group, as revealed by a COSY experiment of Metabolite A. FIG. 2 compares the UV spectra of these components, from diode array UV scan of the respective chromatographic peaks.

FIG. 2 also shows the progression in the peracetylation reaction starting with the Product B-Metabolite A mixture and ending with Product B Acetate/Metabolite A Diacetate, the middle chromatogram having been obtained from the reaction mixture before reaction was complete; it shows unreacted starting materials and some Metabolite A Monoacetate as well.

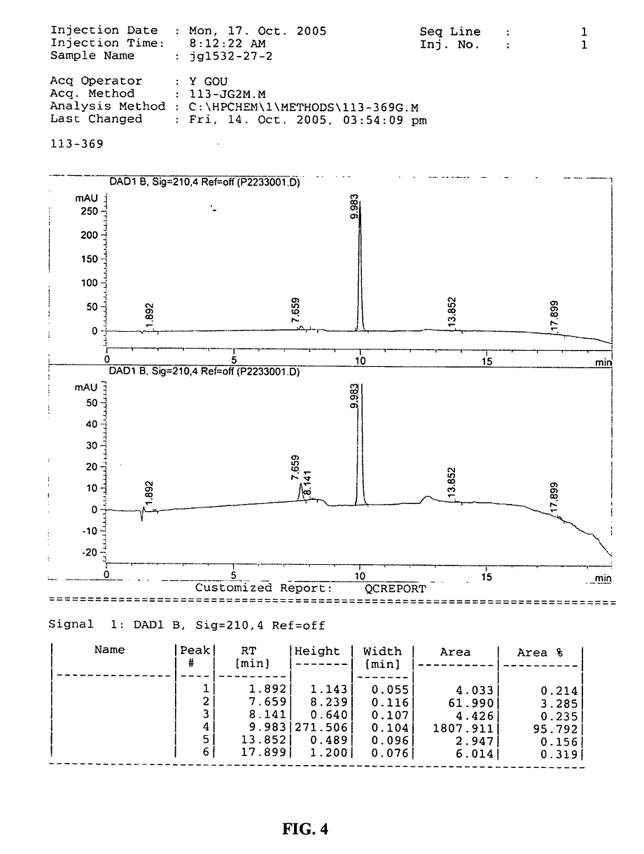

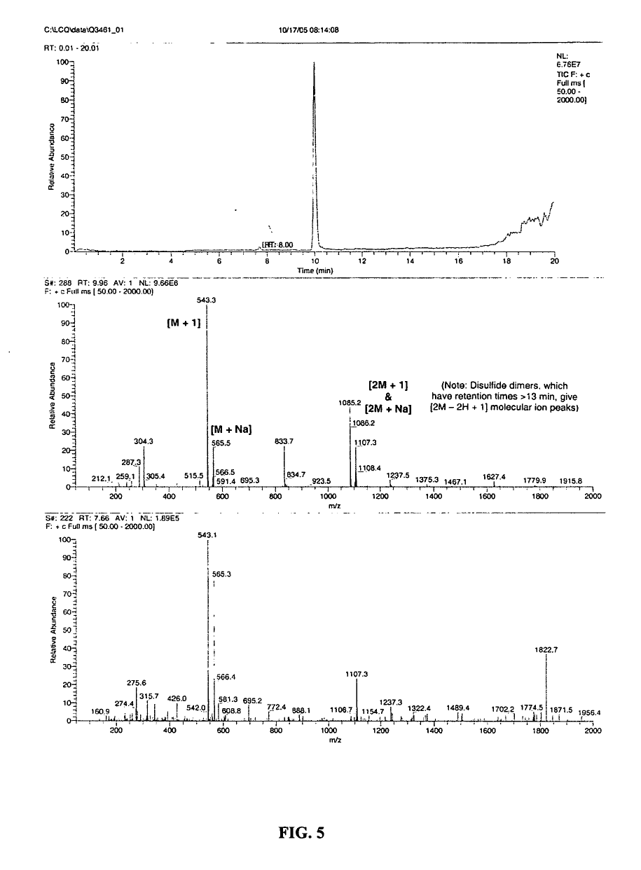

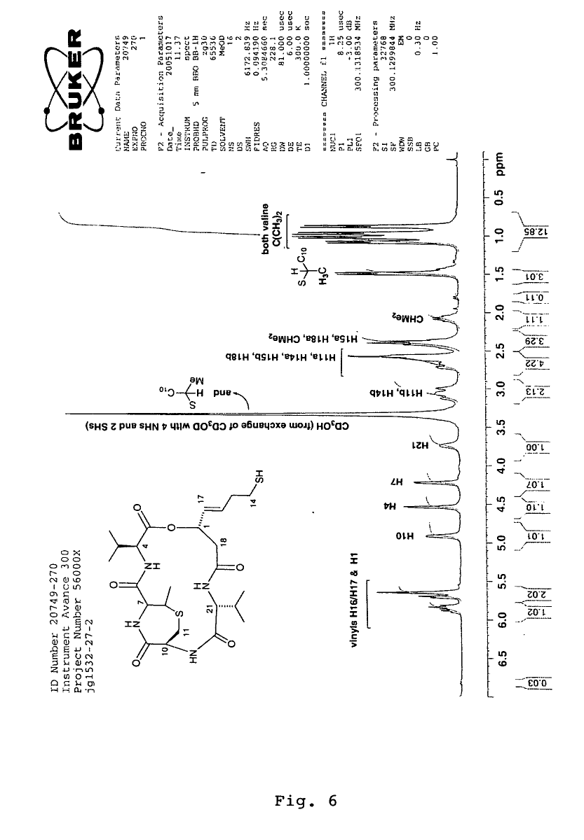

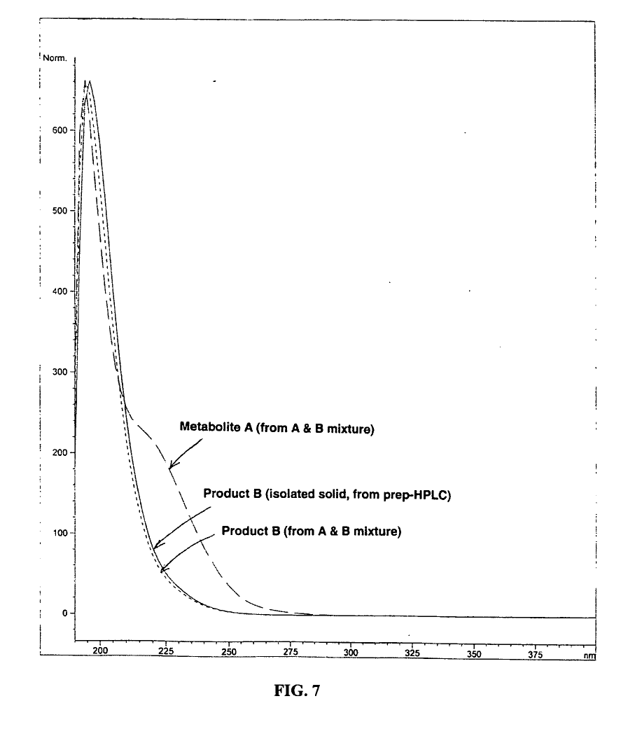

Product B (36 mg; LC purity of 95.7%) was isolated from an equilibrium mixture of Metabolite A and Product B by prep-HPLC. Structure of product B was confirmed NMR, HPLC and UV (see FIGS. 3-7).

### Example

### 2. Thioethers

Formation of Product C Thioethers via Hydride Reduction

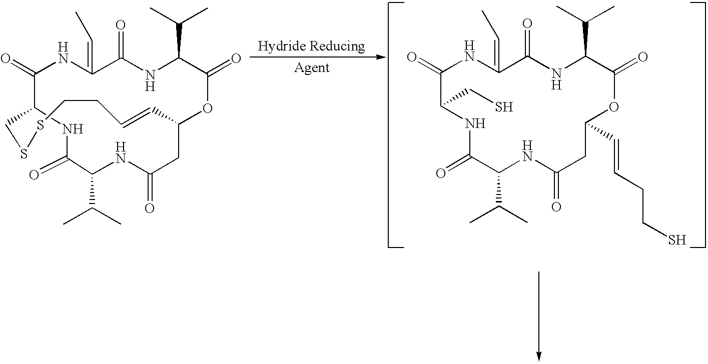

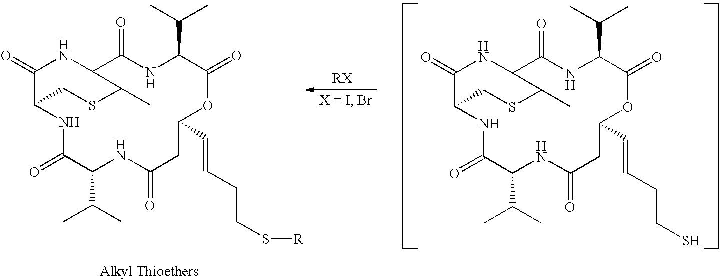

R
CH₃
Benzyl
4-Cl-Benzyl
Butyl
2-Hydroxy ethyl

### Example

### 3. Disulfides

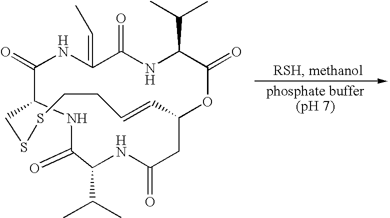 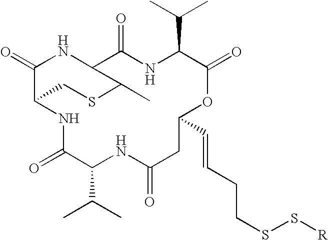 Thiol
R = Methyl
R = Ethyl
R = 2-Hydroxyethyl
R = Isobutyl
R = Phenyl
R = Benzyl
and 4-8 substituted
benzyl and/or phenyl

### Example

### 4. Fatty Acid Disulfides

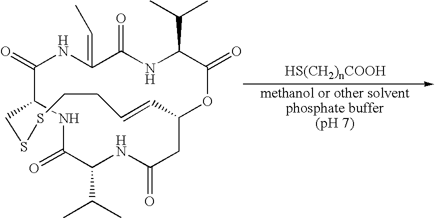 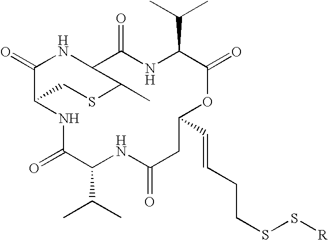 Thiol Fatty Acid
HS(CH₂)₃COOH - butyric
HS(CH₂)₄COOH - pentanoic
HS(CH₂)₅COOH - hexanoic
HS(CH₂)₇COOH - octanoic
HS(CH₂)₉COOH - decanoic

### Example 5

### Amino Acids Disulfides

### Example 6

### FK228 Disulfide Dimer

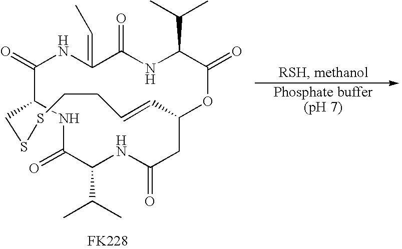

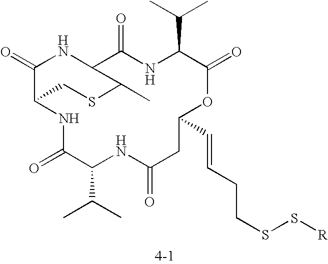

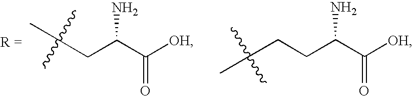

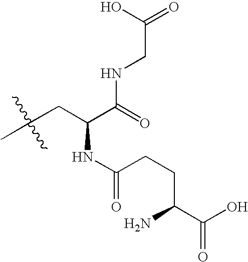

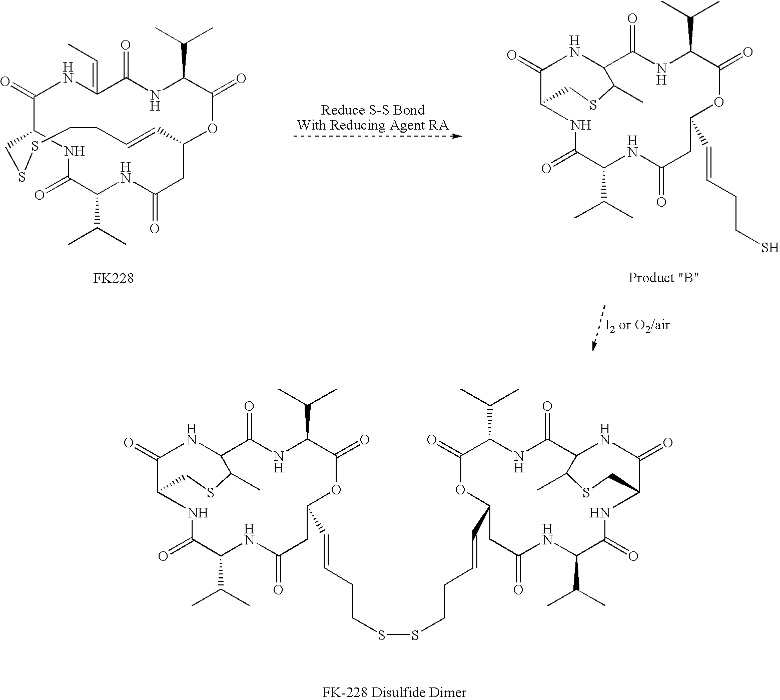

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A compound represented by formula I: 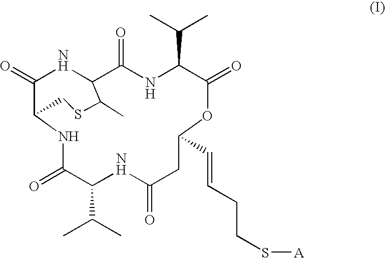
or their racemates, enantiomers, regioisomers, salts, esters or prod rugs thereof, wherein A is a moiety that can be cleaved in vivo to release a thiol group.

2. A compound of claim 1, wherein A is a substituted or unsubstituted, saturated or unsaturated aliphatic group, —COR₁, —SC(═O)—O—R₁ or —SR₂, wherein R₁ is independently hydrogen, a substituted or unsubstituted amino, a substituted or unsubstituted, a saturated or unsaturated aliphatic group, a substituted or unsubstituted, a saturated or unsaturated alicyclic group, a substituted or unsubstituted aromatic group, a substituted or unsubstituted heteroaromatic group, or a substituted or unsubstituted heterocyclic group and R₂ is a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, or a substituted or unsubstituted heterocyclic group.

3. A compound of claim 2, wherein A is alkyl, substituted alkyl, benzyl, substituted benzyl, phenyl, substituted phenyl, provided that A is not a methyl.

4. A compound of claim 2, wherein A is —COR₁ and R₁ is hydrogen, alkyl, benzyl, substituted benzyl, phenyl, substituted phenyl.

5. A compound of claim 2, wherein A is —SC(═O)—O—R₁, and R₁ is substituted or unsubstituted alkyl, or substituted or unsubstituted aromatic group.

6. A compound of claim 2, wherein A is —SR₂ and R₂ is methyl, ethyl, 2-hydroxyethyl, isobutyl, benzyl, substituted benzyl, phenyl, a substituted phenyl, —C₄-C₁₀ acid, cysteine, homocysteine or glutathione.

7. A disulfide dimer of FK228.

8. A pharmaceutical composition comprising a compound of claim 1 or a pharmaceutically acceptable salt, ester or prodrug thereof, in combination with a pharmaceutically acceptable carrier.

9. A method of treating an HDAC-mediated disease comprising administering to a patient in need thereof a pharmaceutical composition of claim 8.

10. A method of treating a proliferative disease comprising administering to a patient in need thereof a pharmaceutical composition of claim 8.

11. A method of treating cancer in a patient comprising administering to a patient in need thereof a pharmaceutical composition of claim 8.

12-14. (canceled)
